(19) United States
(12) Patent Application Publication
(10) Pub. No.: US 2026/0258087 A1
(43) Pub. Date: Sep. 3, 2026

(54) PHARMACEUTICAL COMPOSITION FOR PREVENTING OR TREATING HEPATITIS B

(71) Applicants: GWANGJU INSTITUTE OF SCIENCE AND TECHNOLOGY, Gwangju (KR); SEOUL NATIONAL UNIVERSITY HOSPITAL, Seoul (KR); SEOUL NATIONAL UNIVERSITY R&DB FOUNDATION, Seoul (KR); NATIONAL CANCER CENTER, Gyeonggi-do (KR)

(72) Inventors: JI WON SEO, Gwangju (KR); YONG CHUL KIM, Gwangju (KR); DONG JAE LEE, Gwangju (KR); JI EUN CHOI, Gwangju (KR); YOON JUN KIM, Seoul (KR); SUNG GYOO PARK, Seoul (KR); YU RI CHO, Gyeonggi-do (KR)

(21) Appl. No.: 18/864,640

(22) PCT Filed: May 8, 2023
(86) PCT No.: PCT/KR2023/006216

§ 371 Date: Nov. 11, 2024

(30) Foreign Application Priority Data
May 9, 2022 (KR) 10-2022-0056707

(51) Int. Cl. C07K 7/64; A61K 38/00; A61K 45/06; A61P 31/20

(52) U.S. Cl. CPC C07K 7/64; A61K 45/06; A61P 31/20; A61K 38/00

(57) ABSTRACT

A composition for preventing or treating hepatitis B includes a compound represented by Formula 1 or a pharmaceutically acceptable salt thereof. The composition has excellent bioavailability and has no immunosuppressive and nephrotoxic side effects, and thus can be useful for preventing or treating hepatitis B, and the effect thereof can be further increased through co-administration with other hepatitis B therapeutic agents.

A sequence listing electronically submitted on Nov. 11, 2024 as a XML file named 20241111_LC0952424_TU_SEQ.XML, created on Nov. 1, 2024 and having a size of 3,532 bytes, is incorporated herein by reference in its entirety.

### BACKGROUND

### 1. Technical Field

The present invention relates to a pharmaceutical composition for preventing or treating hepatitis B.

### 2. Background Art

Hepatitis B virus (HBV) is a virus belonging to the family Hepadnaviridae that causes acute or chronic liver disease.

Chronic hepatitis B (CHB) is a globally spreading infectious liver disease caused by the hepatitis B virus (HBV). The HBV invades liver cells, replicates itself, and causes inflammatory damage to infected liver cells. Furthermore, it causes hepatocellular fibrosis, cirrhosis, and carcinoma. About 250 million people suffer from liver dysfunction due to HBV infection. Nucleoside analog antiviral drugs such as lamivudine, entecavir, and tenofovir, which inhibit reverse transcription of the virus, are being used as therapeutic agents.

However, antiviral drugs inhibiting the reverse transcription of the hepatitis B virus do not suppress the entry of the hepatitis B virus into hepatocytes and have the problem of not removing the virus that has infiltrated the liver cells.

### SUMMARY

An object of the present invention is to provide a pharmaceutical composition for preventing or treating hepatitis B.

1. A pharmaceutical composition for preventing or treating hepatitis B, including a compound represented by Formula 1 below or a pharmaceutically acceptable salt thereof:

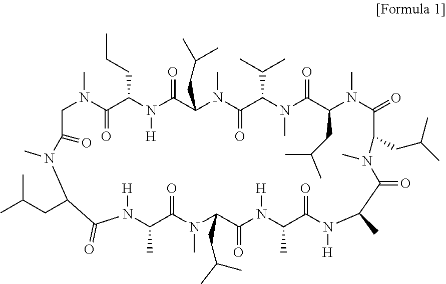 [SMILES: CCC[C@@H]1NC(=O)[C@H](CC(C)C)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C)NC(=O)C(CC(C)C)N(C)C(=O)CN(C)C1=O]

2. The pharmaceutical composition according to the above 1, wherein the hepatitis B is chronic or acute hepatitis B.

3. The pharmaceutical composition according to the above 1, wherein the composition is administered orally, subcutaneously, intramuscular or intravenously.

4. The pharmaceutical composition according to the above 1, further including an HBV antiviral agent or an immunomodulator.

The pharmaceutical composition of the present invention is excellent in effects of preventing or treating hepatitis B. Further, the pharmaceutical composition of the present invention may be used as an effective therapeutic agent for hepatitis B without nephrotoxicity and immunosuppressive side effects.

### BRIEF DESCRIPTION OF DRAWINGS

FIG. 1 illustrates results of intravenous administration (iv, 5 mg/kg, ▴) and oral administration (po, 20 mg/kg, ▪) of CsA or CsO to male ICR mice to evaluate in vivo pharmacokinetic profile. CsA: po, Tₘₐₓ=1.0 h, AUC_{∞,po}=2900±100 ng·h/mL, Cₘₐₓ=540±70 ng/mL, CL=1.17±6 mL/min/kg, V_{z}=46.7±0.6 L/kg, t_{1/2}=4.6±0.3 h, F=NA; CsO: iv, AUC_{∞,iv}=8500±400 ng/h/mL, CL=9.8±0.5 mL/min/kg, Vₛₛ=4.0±0.4 L/kg, t_{1/2}=4.8±0.6 h; po, Tₘₐₓ=0.8±0.1 h, AUC_{∞,po}=4080±50 ng·h/mL, Cₘₐₓ=1600±100 ng/mL, CL=82±1 mL/min/kg, V_{z}=22.2±0.7 L/kg, t_{1/2}=3.1±0.1 h, F=12±0.3%. NA, not applicable.

FIG. 2 shows blood-to-plasma ratios according to the administered concentration of CsA or CsO. Error bars represent standard deviation, and statistical significance was determined using Student's t test (*p<0.05).

FIG. 3 illustrates results of the cyclophilin A binding assay and the catalytic cis-trans isomerization rate of CsA and CsO—inhibitor concentration curve. Error bars represent standard deviation.

FIG. 4 illustrates cytotoxicity test results of CsA and CsO. Each graph represents survival rates of MDCK and BSC-1 cell lines according to the concentration of CsA and CsO.

FIGS. 5A to 5F and FIG. 6 illustrate results of evaluating in vivo HBV entry inhibition efficacy of CsO using a humanized mouse model, wherein FIG. 5A shows serum HBV DNA, FIG. 5B shows covertly closed circular DNA (cccDNA) in hepatocytes, FIG. 5C shows HBsAg, FIG. 5D shows HBeAg, FIG. 5E shows alanine transaminase (ALT), and FIG. 5F shows serum albumin levels. CsO was administered orally at 30 mg/kg daily. Control is a negative control in which no drug was administered. FIG. 6 illustrates results of sectioning a liver tissue sample from a humanized mouse model administered CsO and performing immunohistochemical staining for Hepatitis B core antigen (HBc) and Hepatitis B surface antigen (HBs). HBc is the nuclear antigen of hepatitis B virus, and HBs is the surface antigen of hepatitis B virus. The image was enlarged 20 times.

### DETAILED DESCRIPTION

The present invention relates to a pharmaceutical composition for preventing or treating hepatitis B.

The present invention provides a pharmaceutical composition for preventing or treating hepatitis B, which includes a compound represented by Formula 1 below or a pharmaceutically acceptable salt thereof:

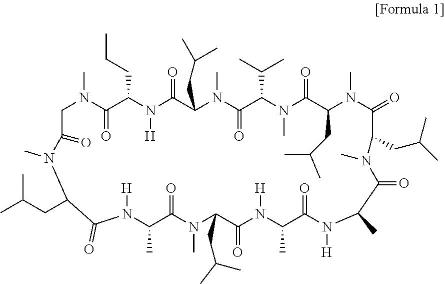 [SMILES: CCC[C@@H]1NC(=O)[C@H](CC(C)C)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C)NC(=O)C(CC(C)C)N(C)C(=O)CN(C)C1=O]

in the structural formula (chemical formula) of this specification, if any substituent is not indicated in a site although the site needs a substituent, it means that a hydrogen substituent is omitted, which would be applied to all structural formulae in the present invention.

The hepatitis B includes both acute hepatitis B and chronic hepatitis B.

The compound represented by Formula 1 may inhibit the entry of hepatitis K virus into liver cells, thereby preventing or treating hepatitis B.

The compound represented by Formula 1 shows high plasma concentration when administered in vivo and has excellent oral bioavailability, such that it can effectively inhibit the entry of hepatitis B virus (HBV) into liver cells even when administered orally.

The compound represented by Formula 1 exhibits low nephrotoxicity and does not cause immunosuppression in the body due to its low binding ability with CypA. Therefore, the pharmaceutical composition including the compound represented by Formula 1 can be used as a therapeutic agent for hepatitis B without nephrotoxicity and immunosuppressive side effects.

The “pharmaceutically acceptable salts” as used herein include salts of the active compounds prepared with relatively non-toxic acids and bases depending on the specific substituents found in the compounds mentioned herein. When the compounds of the present invention have relatively acidic functionality, base addition salts can be obtained by contacting the neutral form of these compounds with a sufficient amount of the desired base, a pure or suitable inert solvent. Examples of pharmaceutically acceptable base addition salts include sodium, potassium, calcium, ammonium, organic amino, or magnesium salts or similar salts. When the compounds of the invention have relatively basic functionality, acidic addition salts can be obtained by contacting the neutral form of these compounds with a sufficient amount of the desired acid, a pure or suitable inert solvent. Examples of pharmaceutically acceptable acidic addition salts include salts derived from non-toxic organic acids such as acetic acid, propionic acid, isobutyric acid, oxalic acid, maleic acid, malonic acid, benzoic acid, succinic acid, suberic acid, fumaric acid, mandelic acid, phthalic acid, benzenesulfonic acid, p-tolylsulfonic acid, citric acid, tartaric acid, methanesulfonic acid, and analogs thereof, as well as hydrogen chloride, hydrogen bromide, nitric acid, carbonic acid, monohydrogencarbonic acid, phosphoric acid, monohydrogencarbonic acid, dihydrogenphosphoric acid, sulfuric acid, monohydrosulfuric acid, hydrogen iodide or phosphorous acid, and analogs thereof. Further, the pharmaceutically acceptable acidic addition salts include salts of amino acids, such as alginate and analogs thereof, and analogs of organic acids, such as glucuronic or galactunoric acids and analogs thereof (e.g., Berge et al. (1977) J. Pharm. Sci. 66: 1-19). Some specific compounds of the invention have both basic and acidic functionality, allowing the compounds to be converted to basic or acidic addition salts. Other examples of the salts can be found in literature known in the art, such as Remington's Pharmaceutical Sciences, 18th eds., Mack Publishing, Easton PA (1990) or Remington: The Science and Practice of Pharmacy, 19th eds., Mack. Publishing, Easton PA (1995).

Suitable subjects to be treated according to the compositions of the present invention include mammalian subjects. Mammals according to the present invention may include, without limitation thereof, humans, dogs (canines), cats (felines), bovines, goats (caprines), horses (equine), sheep (ovine), pigs (porcines), rodents, lagomorphs, primates, etc., includes mammals in utero, and may specifically be humans. The subject may be of amphophilic and may be at any stage of development.

The compound according to the invention may be administered by any suitable route, in the form of a pharmaceutical composition suitable for such route, and in a dosage effective for the intended treatment. The effective dosage is generally about 0.001 to about 100 mg/kg of body weight/day, preferably about 0.01 to about 30 mg/kg/day, in single or divided doses. Depending on age, species, and disease or condition to be treated, dosage levels below the lower limit of this range may be appropriate. In other cases, still larger doses may be used without harmful side effects. Larger doses may be divided into several smaller doses for administration throughout the day. Methods for determining appropriate dosages are well known in the art, and may use, for example, Remington: The Science and Practice of Pharmacy, Mack Publishing Co., 20th ed., 2000.

The pharmaceutical composition according to the present invention may be administered as follows.

The pharmaceutical composition according to the present invention may be administered orally (oral administration), and the oral cavity is a concept including swallowing. By oral administration, the compounds of the invention may enter the gastrointestinal tract or be absorbed directly from the mouth into the bloodstream, for example, like buccal or sublingual administration.

Compositions suitable for oral administration may be in the form of solid, liquid, gel, or powder, and may have formulations such as tablets, lozenges, capsules, granules, powder and the like.

The composition for oral administration may optionally be enteric coated, and may exhibit delayed or sustained release through the enteric coating. That is, the composition for oral administration according to the present invention may be in a formulation with an immediate or modified release pattern.

Liquid formulations may include solutions, syrups, and suspensions, and these liquid compositions may be contained in soft or hard capsules. These formulations may include pharmaceutically acceptable carriers such as water, ethanol, polyethylene glycol, cellulose, or oil. The formulation may also include one or more emulsifying agents and/or suspending agents.

In tablet formulations, an amount of drug as the active ingredient may be present in an amount of about 0.05 to 95% by weight (“wt. %”) relative to a total weight of the tablet, more typically about 2 to 50 wt. % of the formulation. In addition, the tablets may contain disintegrants in an amount of about 0.5 to about 35 wt. %, more typically about 2 to 25 wt. % of the formulation. Examples of disintegrants may include lactose, starch, sodium starch glycolate, crospovidone, croscarmellose sodium, maltodextrin, or a mixture thereof, but they are not limited thereto.

Suitable lubricants included for preparation into tablets may be present in an amount of about 0.1 to 5 wt. %, and talc, silicon dioxide, stearic acid, calcium, zinc or magnesium stearate, sodium stearyl fumarate, etc. may be used as the lubricant, but in the present invention, they are not limited thereto in terms of the types of these additives.

Binders used for preparation into tablets may include gelatin, polyethylene glycol, sugar, gum, starch, polyvinylpyrrolidone, hydroxypropylcellulose, hydroxypropylmethylcellulose and the like. Suitable diluents used for preparation into tablets may include mannitol, zylitol, lactose, dextrose, sucrose, sorbitol, starch, microcrystalline cellulose, and the like, but in the present invention, they are not limited thereto in terms of the types of these additives.

Solubilizers that can be optionally included in the tablets may be used in an amount of about 0.1 to 3 wt. % based on the total weight of the tablet, and may include, for example, polysorbate, sodium lauryl sulfate, sodium dodecyl sulfate, propylene carbonate, diethyleneglycol monoethylether, dimethyl isosorbide, polyoxyethyleneglycolated natural or hydrogenated castor oil, HCOR™ (Nikkol), oleyl ester, GELUCIRE™, caprylic/caprylic acid mono/diglycerides, sorbitan fatty acid ester, SOLUTOL HS™, and the like in the pharmaceutical composition according to the present invention, but in the present invention, they are not limited thereto in terms of the specific types of these solubilizers.

The pharmaceutical composition according to the present invention may be administered directly into subcutaneous tissue, bloodstream, muscle or intestine. Suitable methods for parenteral administration may include intravenous, intramuscular, subcutaneous intraarterial, intraperitoneal, intrathecal, and intracranial injections. Suitable devices for parenteral administration may include injectors (including needles and needleless syringes) and infusion methods.

Compositions for parenteral administration may be formulations with an immediate or modified release pattern, which may be a delayed or sustained release pattern.

Most of parenteral formulations are liquid compositions, and these liquid compositions are aqueous solutions including medicinal ingredients, salts, buffers, isotonic agents, etc. according to the present invention.

The parenteral formulations may also be prepared in dried form (e.g., lyophilized) or as sterile non-aqueous solutions. These formulations may be used together with a suitable vehicle such as sterile water. Solubility-enhancing agents may also be used in the preparation of parenteral solutions.

The pharmaceutical composition according to the present invention may be administered topically to the skin or transdermally. Formulations for topical administration may include lotions, solutions, creams, gels, hydrogels, ointments, foams, implants, patches and the like. Pharmaceutically acceptable carriers for topical administration formulations may include water, alcohol, mineral oil, glycerin, polyethylene glycol and the like. The topical administration may also be accomplished by electroporation, iontophoresis, phonophoresis and the like.

Compositions for topical administration may be formulations with an immediate or modified release pattern, which may be a delayed or sustained release pattern.

Methods for preparing pharmaceutical compositions for appropriate treatment or prevention of diseases or conditions are well known to persons having common knowledge in the technical field to which the present invention pertains. For example, as described in Handbook of Pharmaceutical Excipients (7th ed.), Remington: The Science and Practice of Pharmacy (20th ed.), Encyclopedia of Pharmaceutical Technology (3rd ed.), Sustained and Controlled Release Drug Delivery Systems (1978), etc., a pharmaceutical composition for the purpose of the present invention can be prepared by appropriately mixing pharmaceutically acceptable carriers, carriers, additives, etc. with the compound according to the present invention.

The pharmaceutical composition according to the present invention may be used alone or in combination with other pharmaceutically active compounds to prevent or treat hepatitis B. The compound according to the invention and other pharmaceutically active compound(s) may be administered simultaneously (in the same formulation or in separate formulations) or sequentially.

The one or more additional pharmaceutically active compounds may be known antiviral agents or immunomodulators against hepatitis B virus (HBV). The antiviral agents may include, for example, HBV capsid assembly inhibitors, surface antigen (HbsAg) secretion inhibitors, ribonucleic acid inhibitors (RNA interference and antisense oligonucleotides), and nucleoside analogs and the like. The immunomodulators may include, for example, PEGylated interferon, interferon alpha and the like.

The pharmaceutical composition of the present invention may further include one or more additional pharmaceutically active compounds other than the compound according to the present invention.

One or more additional pharmaceutically active compounds are as described above.

Hereinafter, the present invention will be described in more detail through examples.

### Example 1. Evaluation of Permeability Using PAMPA

Parallel artificial membrane permeability assay (PAMPA) was performed to evaluate the permeability of Carbamazepine (Cz), Cyclosporine A (CsA), and Cyclosporine C (the compound represented by Formula 1 of the present invention, CsO) under passive diffusion. From the results, it was confirmed that CsO showed 7 times lower permeability than CsA (Table 1).

TABLE 1

| | Permeability | | |
|---|---|---|---|
| Compound | (×10⁻⁶ cm/s)^{a} | R (%)^{b} | AlogP^{c} |
| Cz | 11 ± 1 | | |
| CsA | 1.97 ± 0.23 | 87 ± 1 | 4.333 |
| CsO | 0.29 ± 0.09 | 84 ± 9 | 4.765 |

^{a}permeability was determined by PAMPA,
^{b}R(%): mass retention,
^{c}AlogP was calculated using Discovery Studio v.17.1, Cz: carbamazepine.

### Example 2. Evaluation of Cell Membrane Permeability

In vitro membrane permeability of CsO was evaluated using Caco-2 cells. Caco-2 cell monolayer is a cell line used to represent the intestinal epithelium in order to evaluate the apparent permeability of drugs. Drugs can permeate the Caco-2 cell monolayer by the transcellular route, paracellular route, or active transporters.

Caco-2 permeability assay was performed in triplicate. A 10 μM sample was dissolved in 1% (v/v) DMSO/HBSS solution, and 100 μM verapamil in Hank's Balanced Salt Solution (HBSS) buffer was used as a P-glycoprotein inhibitor. Inserts with Caco-2 cells were pre-incubated with verapamil for 30 min at 37° C., and then incubated for 2 hours at 37° C. without shaking. Efflux ratio was calculated as A to B permeability/B to A permeability. Aliquots (20 μL) were collected at each time point (0.5, 1, 1.5, and 2 hours). Experiments were performed in duplicate.

Caco-2 membrane permeability of CsA and CsO was consistent with the results of membrane permeability by PAMPA. These results indicate that CsA and CsO permeate the cell membrane by the intercellular route under the same mechanism, i.e., passive diffusion (Table 2).

TABLE 2

| | Caco-2 permeability assay | | | | | | |
|---|---|---|---|---|---|---|---|
| | | | | | | A to B with | |
| | A to B | | B to A | | | verapamil | |
| | Permeability | | Permeability | | Efflux | Permeability | |
| | (×10⁻⁶ | % | (×10⁻⁶ | % | ratio | (×10⁻⁶ | % |
| Compound | cm/s) | recovery | cm/s) | recovery | (ER) | cm/s) | recovery |
| CsA | 1.51 ± 0.30 | 58 ± 6 | 4.51 ± 1.14* | 52 ± 1* | 3 | 2.50 ± 0.30 | 68 ± 6 |
| CsO | 0.29 ± 0.05 | 82 ± 3 | 2.88 ± 0.97 | 82 ± 17 | 10 | 0.69 ± 0.19 | 123 ± 3 |
| Propranolol | 20.0 ± 2.7 | 121 ± 8 | 20.4 ± 9.3* | 157 ± 2* | 1 | 25.7 ± 4.5 | 101 ± 7 |
| loperamide | 2.9 ± 2.0 | 88 ± 6 | 27.5 ± 1.6 | 85 ± 1 | 10 | 5.1 ± 1.1 | 78 ± 1 |

### Example 3. In Vivo Pharmacokinetic Study

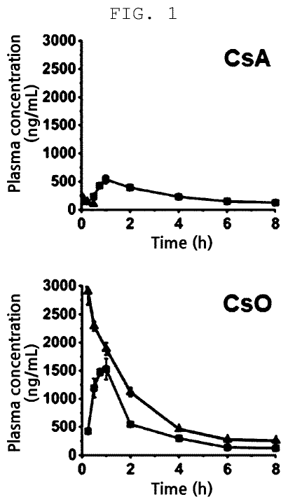

To evaluate the in vivo pharmacokinetic profile, CsA or CsO was injected intravenously (i.v, 5 mg/kg, ▴) or orally (po, 20 mg/kg, ▪) into male ICR mice (FIG. 1).

After oral administration, CsO and CsA were rapidly absorbed and exhibited almost identical Tmax, thereby indicating absorption by the same mechanism (FIG. 1). CsO showed moderate oral bioavailability (F=12±0.3). However, oral bioavailability of CsA could not be achieved due to low plasma concentration after intravenous injection.

CsO showed higher plasma concentration than CsA after intravenous injection. In addition, the plasma concentration of CsA fell below the lower limit of quantification 15 minutes after injection.

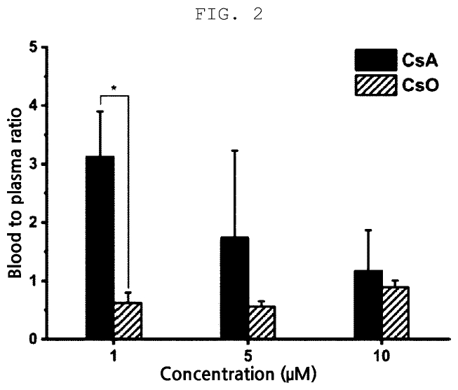

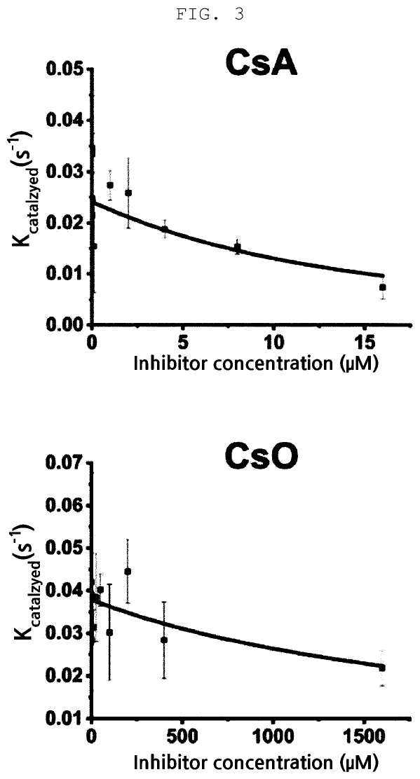

In the blood to plasma partitioning assay (BPP), CsA showed a blood-to-plasma partitioning ratio (BPR) that was 5 times higher than that of CsO at a dose of 1 μM (CsA=3.12, CsO=0.62, see FIG. 2). As the dose was increased, the BPR for CsO was increased (FIG. 2). The high BPR of CsA indicates that CsA is preferentially accumulated in the erythrocytes, thereby resulting in lower plasma concentration while the low BPR of CsO (BPR <1 at all doses) indicates that CsO is preferentially accumulated in the plasma, thereby resulting in high plasma concentration.

### Example 4. Binding Ability of CsO with Cyclophilin A

The inhibition constant (Ki) against CypA was measured using an uncoupled peptidyl-prolyl isomerase functional assay.

Table 3 shows the inhibitory constant (Kᵢ) of CsA and CsO against cyclophilin A (CypA).

TABLE 3

| Compound | Kᵢ (μM) |
|---|---|
| CsA | 0.007 ± 0.003 |
| CsO | 2.3 ± 0.9 |

CsO was found to have a binding inhibitory constant against CypA that was more than 300 times higher than that of CsA. CypA is a substance that is especially over-expressed in red blood cells. CsA is preferentially accumulated in the red blood cells due to binding with CypA, thereby resulting in low plasma concentration. CsO is preferentially accumulated in the plasma due to low binding ability with CypA, thereby resulting in high plasma concentration, which is also consistent with the clinical pharmacokinetic profile results.

### Example 5. Cytotoxicity of CsA and CsO on Renal Epithelial Cell Line

MDCK and BCS-1 cells were purchased from Korean Cell Link Bank. MDCK and BCS-1 cells were grown in DMEM medium containing 10% FBS at 37° C. with 5% CO₂. MTS cell-viability assay was performed to evaluate the effect of cyclic peptides on cell growth. Aliquots (100 μL) of medium containing 1×10⁵ cells were poured into each well of a 96-well plate (SPL). The cells were allowed to grow and attach in a humidified atmosphere of 5% CO₂ in air at 37° C. for 24 hours.

When the cell density reached about 70=confluency, the medium was replaced with serial dilutions of cyclic peptide stock solution (10 mM in DMSO) and incubated for 24 hours. After aspirating the medium, Opti-MEM solution (200 μL) including CellTiter 96 aqueous non-radioactive cell proliferation assay reagent (Promega, Madison, WI, USA) containing tetrazolium compound [3-(4,5-dimethylthiazol-2-yl)-5-(3-carboxymethoxyphenyl)-2-(4-sulfophenyl)-2H-tetrazolium, inner salt; MTS](20 μL) was added to each well. Plates were incubated at 37° C. for 4 hours for metabolism. MTS-formazan product in viable cells was measured at 490 nm using a microplate reader, and cell viability (i) was determined and compared with untreated cells. Percentage of cell viability=(A−A_{blank})/(A_{control}−A_{blank})×100, wherein A is the absorbance of wells including cells treated with cyclosporine, and A_{control} is the wells including cells not treated with cyclosporine. The average absorbance, A_{blank}, is the background absorbance in the absence of cells.

As a result, it was confirmed that CsO showed lower toxicity than CsA to renal epithelial cell lines (see Table 4 and FIG. 4).

Table 4 shows LC₅₀ values of CsA and CsO for eukaryotic cell lines. BSC-1 and MDCK are monkey and canine kidney epithelial cells, respectively, and HeLa is human cervical tumor cells. In Table 4, a: percentage cell viability at 200 μM, and b: percentage cell viability at 100 μM.

TABLE 4

LC₅₀ (μM)

| Cell line | CsA | CsO |
|---|---|---|
| BSC-1 | >200 (62.4 ± 0.4%)^{a} | >200 (76 ± 2%)^{a} |
| MDCK | 15.2 | 19.9 |
| HeLa | >100 (82 ± 5%)^{b} | >100 (84 ± 3%)^{b} |

### Example 5. Confirmation of In Vitro HBV Host Invasion Inhibition Effect

HepG2 cells are representative cells that have immortalized human liver cells, and HepG2-NTCP cells refer to cells that overexpress sodium-taurocholate cotransporting polypeptide (NTCP). HepG2-NTCP cells are representative cells used to confirm the effect of inhibiting HBV host invasion.

HepG2-NTCP cells were grown in DMEM medium containing 10% FBS, penicillin (100 U), and streptomycin (0.1 mg/mL) at 37° C. with 52 CO₂. As a result of conducting a gene amplification test on these cells, they were found to be negative for Mycoplasma bacteria. The HepG2-NTCP cells were incubated with cyclic peptides in 12-well plates for 2 hours. Afterwards, the cells were infected by treating HBV particles (2×10⁶ virus/mL) and cyclic peptide together and cultured for 3 hours. After removing the medium containing the virus, the cells were cultured for 16 hours. After the cells were treated with cyclic peptide, intracellular HBV DNA was extracted, purified, and quantified using a gene amplification test. To isolate HBV DNA from host cells, cells were lysed with cell lysis buffer (50 mM Tris-HCl, pH 7.5, 1 mM EDTA, 1% NP40) for 10 minutes on ice. After removing cell debris and nuclei by centrifugation at 15,000 times gravity, the supernatant was treated with micrococcal nuclease (0.25 U/μL) at 37° C. for 1 hour. Micrococcal nuclease was inactivated by adding EGTA to a final concentration of 10 nM. Afterwards, intracellular DNA of HBV was extracted using the G-SPIN™ set. The extracted intracellular DNA of HBV was amplified using HBV DNA primers (forward: TCCTCTTCATCCTGCTGCTATG (SEQ ID NO: 1), reverse: CGTGCTGGTAGTTGATGTTCCT (SEQ ID NO: 2)) and probe (TATTGGTTCCTTGGACTA (SEQ ID NO: 3)).

In vitro HBV entry inhibition assay based on quantification of intracellular DNA of HBV was performed. CsA and CsO showed comparable entry inhibition rates at 1 μM concentration (Table 5). The IC₅₀ measurement results showed that CsO had an IC₅₀ value similar to that of CsA, thereby demonstrating great efficacy in inhibiting HBV infection (IC₅₀=0.51±0.05 μM) (Table 6).

Table 5 shows results of in vitro HBV entry inhibition screening. Each cyclosporine A or O was used at a concentration of 1 μM to evaluate the HBV entry inhibitory activity. The experiment was performed in triplicate (n=3).

TABLE 5

| Compound | HBV inhibition (%) |
|---|---|
| CsA | 83 ± 8 |
| CsO | 75 ± 4 |

Table 6 shows IC₅₀ values of CsA and CsO. The IC₅₀ values are expressed as mean±SD. The CsA experiment was performed in six replicates, and the CsO experiment was performed in triplicate.

TABLE 6

| | In vitro HBV entry inhibition |
|---|---|
| Compound | (IC₅₀, μM) |
| CsA | 0.40 ± 0.03 |
| CsO | 0.51 ± 0.05 |

### Example 6. Confirmation of In Vivo HBV Host Invasion Inhibition Effect

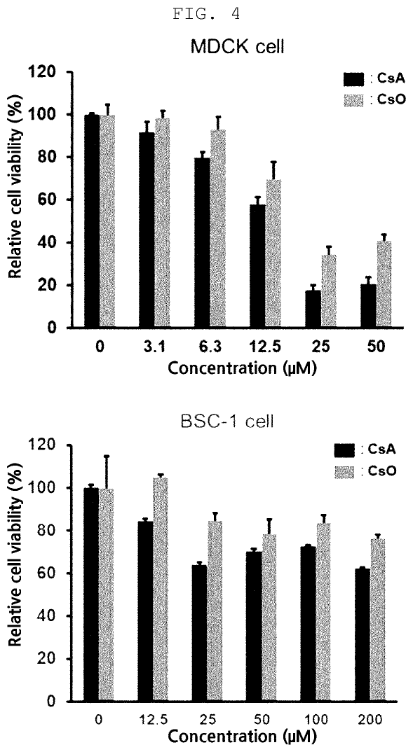

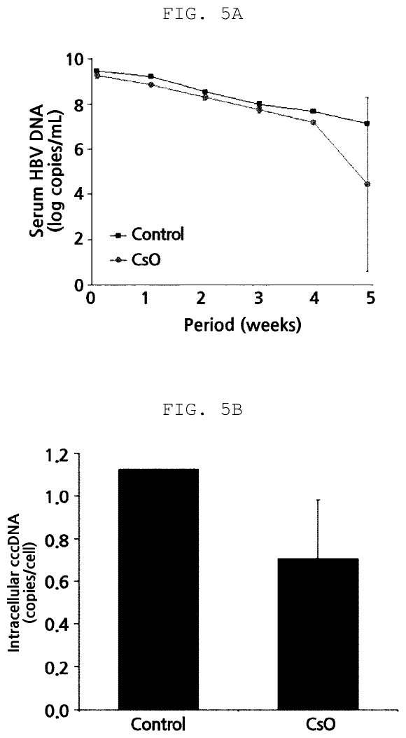

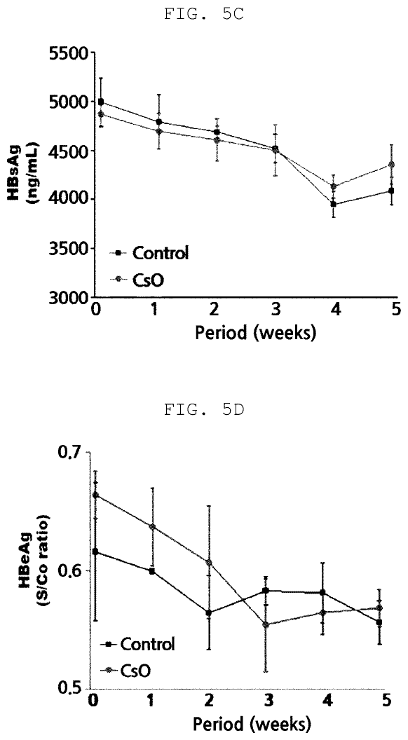

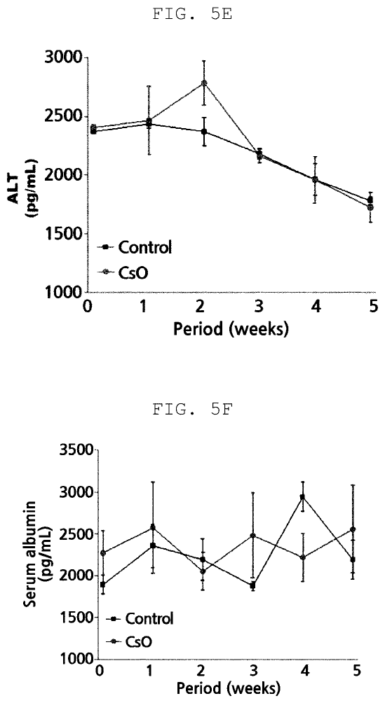

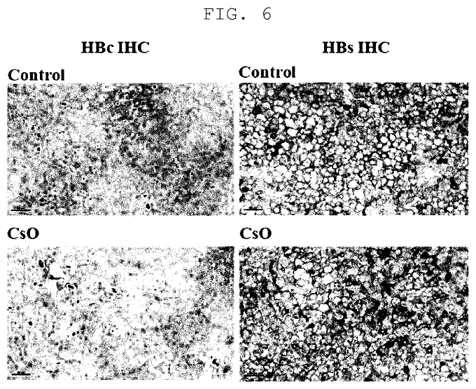

Antiviral activity was measured after administering CsO to a humanized liver mouse model infected with HBV. Human liver chimeric urokinase-type plasminogen activator/severe combined immunodeficiency (uPA/SCID) mice were obtained by transplanting human hepatocytes (10⁵ to 10⁶ cells per mouse) into 3-week-old uPA/SCID mice purchased from PhoenixBio Co. (Higashi Hiroshima, Japan) via the spleen. Nine weeks after transplantation, the substitution index (RI), an indicator of humanization, was high at 90% or more in all mice. Then, 17-week-old mice were injected intravenously once with human HBV virus (5×10⁷ copies per mouse, genotype D). After 5 weeks, persistent HBV replication (infection) was confirmed in the chimeric livers of uPA/SCID mice. Drug treatment was performed at 22 weeks of age, and mice were orally administered with CsO (30 mg/kg) daily. Serum samples were collected every week to measure serum HBV DNA, HBsAg, HBeAg, alanine aminotransferase (ALT) and serum albumin levels, and intracellular cccDNA, which are indicators for evaluating the effectiveness of hepatitis B treatment (FIGS. 5A to 5F). As a result, all the serum HBV DNA, HBsAg, HBeAg, alanine aminotransferase (ALT), and serum albumin levels were reduced, and the serum HBV DNA levels and cccDNA levels in liver cells were significantly reduced in the CsO-treated group compared to the negative control. Through this, the excellent hepatitis B treatment effect of CsO could be confirmed.

Further, after 35 days of drug treatment, mice were euthanized with CO₂, liver tissue samples were sectioned, and immunohistochemical staining was performed with anti-HBV core antibody (QAAI00128, Aviva Systems Biology, USA) (FIG. 6). Then, it was confirmed that HBV antigen was reduced.

1: A composition comprising a compound represented by Formula 1 or a pharmaceutically acceptable salt thereof:

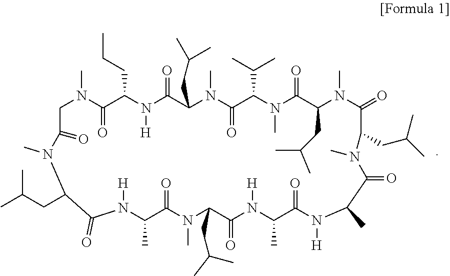 [SMILES: CCC[C@@H]1NC(=O)[C@H](CC(C)C)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C)NC(=O)C(CC(C)C)N(C)C(=O)CN(C)C1=O]

2. (canceled)

3. (canceled)

4: The composition according to claim 1, further comprising an HBV antiviral agent or an immunomodulator.

5: A method for treating hepatitis B or ameliorating a symptom of hepatitis B, the method comprising:
administering to a subject in need thereof a composition comprising a compound represented by Formula 1 below or a pharmaceutically acceptable salt thereof:

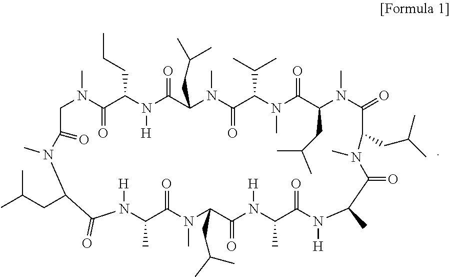 [SMILES: CCC[C@@H]1NC(=O)[C@H](CC(C)C)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C)NC(=O)C(CC(C)C)N(C)C(=O)CN(C)C1=O]

6: The method of claim 5, wherein the hepatitis B is chronic hepatitis B.

7: The method of claim 5, wherein the hepatitis B is acute hepatitis B.

8: The method of claim 5, wherein the composition is administered orally.

9: The method of claim 5, wherein the composition is administered subcutaneously.

10: The method of claim 5, wherein the composition is administered intramuscularly.

11: The method of claim 5, wherein the composition is administered intravenously.

12: The method of claim 5, wherein the composition further comprises an HBV antiviral agent and/or an immunomodulator.


SEQUENCE LISTING (3 sequences)

SEQ ID NO: 1 - DNA, 22 bp, synthetic construct
  source 1..22: mol_type = other DNA
tcctcttcatcctgctgctatg

SEQ ID NO: 2 - DNA, 22 bp, synthetic construct
  source 1..22: mol_type = other DNA
cgtgctggtagttgatgttcct

SEQ ID NO: 3 - DNA, 19 bp, synthetic construct
  source 1..19: mol_type = other DNA
tattggttcttctggacta